(19) United States
(12) Patent Application Publication
(10) Pub. No.: US 2007/0244052 A1
(43) Pub. Date: Oct. 18, 2007

(54) Methods for Identifying Agonists or Antagonists of CD200R Signaling Molecules and Methods of Using the Same

(75) Inventors: Shuli Zhang, Foster City, CA (US); Joseph H. Phillips, Palo Alto, CA (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/772,564

(22) Filed: Jul. 2, 2007

Related U.S. Application Data
(63) Continuation of application No. 11/016,018, filed on Dec. 17, 2004.
(60) Provisional application No. 60/530,967, filed on Dec. 19, 2003.

(51) Int. Cl. A61K 38/00; A61P 31/18

(52) U.S. Cl. 514/12

(57) ABSTRACT

The present invention provides methods for identifying agonists or antagonists of CD200R signaling. In addition, the present invention provides methods for treating patients suffering from CD200R-mediated medical conditions.

This filing is a U.S. Patent Application which claims benefit of U.S. Provisional Patent Application No. 60/530,967, filed Dec. 19, 2003, which is incorporated herein by reference.

### FIELD OF THE INVENTION

The present application relates to methods for identifing agonists or antagonists of CD200R signaling, and methods for treating patients suffering from CD200R-mediated medical conditions.

### BACKGROUND OF THE INVENTION

The immune system comprises complex processes providing rapid responses against evolving pathogens while controlling these same responses to prevent damage to self. These processes include secreted proteins as well as communication between proteins on opposing cell surfaces. In the later case, the cells themselves need to come into contact and the precise location of the cells within tissues is of importance. For example, interaction between CD200 (also known as OX2), a membrane glycoprotein expressed on many cells, and its receptor (CD200R) expressed solely on hematopoietic cells (particularly myeloid cells), has been shown to deliver inhibitory signals to myeloid cells (e.g., mast cells and macrophages). See, Barclay et al., “CD200 and membrane protein interactions in the control of myeloid cells,” Trends Immunol, 23(6):285-290 (2002).

In fact, CD200-deficient mice had observable alterations in the behavior of myeloid cells in tissues that normally expressed CD200, including an increase in number and state of activation of macrophages in several tissues and a profound increase in susceptibility to autoimmune disease models affecting the brain, joints, and retina. These results indicate that CD200-CD200R interactions are involved in the control of mycloid cellular function. See, Hoek, R. M. et al., “Down-regulation of the macrophage lineage through interaction with OX2 (CD200).” Science, 290:1768 (2000); and Dick et al., “Control of myeloid activity during retinal inflammation,” J Leukoc Biol, 74(2):161-166 (2003).

The broad distribution of CD200 and changes in its level of expression provide a mechanism for locally regulating myeloid cellular activity at appropriate sites, such as inflamed tissue. Moreover, the CD200-CD200R regulatory mechanism is an attractive target for immunomodulation because its manipulation can induce immune tolerance and autoimmune diseases. For instance, CD200-Fc fusion proteins have been shown to provide beneficial immunomodulatory effects in models of arthritis and allograft rejection. See, Barclay et al., “CD200 and membrane protein interactions in the control of myeloid cells,” Trends Immunol, 23(6):285-290 (2002).

Elucidation of the mechanism by which CD200-CD200R interaction inhibits myeloid cell function would facilitate the development of methods for identifying agonists or antagonists of CD200R signaling, and methods for treating patients suffering from CD200R-mediated medical conditions.

The present inventors have responded to the above needs by revealing an inhibitory pathway used by CD200R in modulating inhibition of myeloid cell function. In addition, the present inventors have developed methods for identifying agonists or antagonists of CD200R signaling as well as methods for treating patients suffering from CD200R-mediated medical conditions.

### SUMMARY OF THE INVENTION

The present invention is based upon the discovery of the signally pathway utilized by CD200RA to modulate various aspects of immunity. Provided is a method for modulating CD200R signaling in a mammal, comprising administering to the mammal an effective amount of an agent that is effective to modulate CD200R signaling in the mammal. In further embodiments, the CD200R signaling is measured by: assaying TNF-alpha production; or assaying mast cell degranulation in a sample taken from the mammal. It is contemplated that the modulation is enhancement or inhibition of CD200R signaling. It is also provided that the agent is administered at a frequency and for a duration sufficient to inhibit cytokine production in the mammal at a level lesser than the cytokine production measured prior to the step of administering the agent. In another embodiment, the agent is administered at a frequency and for a duration sufficient to enhance cytokine production in the mammal at a level greater than the cytokine production measured prior to the step of administering the agent.

The present invention provides a method for treating CD200-mediated medical conditions in a mammal, comprising administering to the mammal an effective amount of an agonist of CD200R signaling. The agonist binds to a Dok protein

The present invention also provides a method for treating CD200-mediated medical conditions in a mammal, comprising administering to the mammal an effective amount of an antagonist of CD200R signaling. The antagonist binds to a Dok protein.

Also encompassed by the present invention is a method of modulating physiology or development of a cell by contacting the cell with an agonist or antagonist of CD200R. In further embodiments, the agonist or antagonist binds to a Dok protein.

These and other aspects of the present invention will be better appreciated by reference to the following Detailed Description.

### DETAILED DESCRIPTION OF THE INVENTION

The present inventors provide a new inhibitory pathway used by CD200R. Upon ligand or agonist antibody binding, CD200R is phosphorylated on tyrosine and binds to inhibitory adapter proteins Dok1 and Dok2 which in turn are phosphorylated. Next, Dok1 and Dok2 recruit Ras GTPase activating protein (RasGAP). In addition, Dok1 recruits phosphatase SH2-containing inositol phosphatase (SHIP). RasGAP and SHIP mediate the inhibition of the Ras/Raf/mitogen-activated protein kinase (MAPK) pathway that activates extracellular signal-regulated kinase (ERK), c-Jun NH(2)-terminal kinase (JNK), and p38 MAPK as well as TNF-alpha production. Thus, CD200R engagement results in the inibiton of mast cell degranulation and cytokine production.

Most inhibitory receptors on immune cells share a cytoplasmic amino acid sequence, (I/V/L/S)xYxx(L/V), termed the immunoreceptor tyrosine-based inhibitory motif (ITIM). Upon phosphorylation of the tytosine residue in this sequence, ITIM-bearing receptors bind to phosphatase ShpI and/or Ship, and suppress cell activation by promoting dephosphorylation reactions. Unlike most inhibitory receptors (e.g., FcrRIIB, gp49Bl, and CD47), CD200R lacks an ITIM. But, CD200R's cytoplasmic tail contains the amino acid sequence NPxY, a potential phosphotyrosine binding (PTB) domain. The PTB domain is a protein module with 100-170 amino acids that preferentially binds to a NPx(pY) motif. PTB domain proteins include Shc, IRS-1, Dok, RGS12, X11, Fe65, Numb(fly), and Dab(Fly). Interestingly, even though PTB domain proteins share low sequence homology, they exhibit high ligand binding specificity.

Among PTB domain proteins, downstream of tyrosine kinase (Dok) proteins have been shown to mediate inhibitory signaling. The Dok family comprises five known members named Dok1, Dok2 (also known as DokR and FRIP), Dok3 (also known as DokL), Dok4, and Dok5. These molecules contain an amino-terminal pleckstrin homology (PH) domain, a central PTB domain, and a carboxyl-terminal region with multiple potential tyrosine phosphorylation sites and proline-rich regions which may serve as docking sites for Src homology 3 (SH3) domains. Dok1, Dok2, and Dok3 are expressed mainly in hematopoietic cells, while Dok4 and Dok5 are mainly in non-hematopoietic cells.

Dok proteins undergo tyrosine phosphorylation in response to a variety of stimuli, such as immunoreceptor ligation, growth factors, and cytokines. The phosphorylation of Dok proteins triggers phosphotyrosine-binding Src homology 2 (SH2) domain-mediated interactions with inhibitory effectors including Ras GTPase activating protein (RasGAP), SHIP, and Csk (a negative regulator of Src). Such interactions between Dok and these inhibitory effectors leads to inhibition of stimuli, for example, as would normally result from (BCR) signaling.

Dok1 negatively regulates MAPK activation and cell proliferation upon coaggregation of B-cell receptor and FcRIIB. Dok2 negatively regulates T-cell development by recruiting RasGAP and Nck. In mast cells, co-crosslinking of Fc-gamma-RIIB with Fc-epsilon-RI stimulates Dok1 tyrosine phosphorylation and subsequent association with SHIP and RasGAP. Overexpression of Dok1 in mast cells line RBL-2H3 inhibited Fc-epsilon-RI-mediated Ras/Raf/Erk signaling and the de novo synthesis of TNF-alpha. These studies have established that Dok family proteins are inibitory adapter molecules, presumably due to their ability to recruit inhibitory effectors RasGAP, SHIP, and Csk.

### DEFINITIONS

To provide a better appreciation of the present invention, the following terms are defined.

As used herein the phrase “CD200R-mediated medical condition” refers to any state where modulation of Ras/Raf/MAPK signaling would ameliorate that state. Modulation of CD200R signaling may occur through CD200-CD200R interaction as well as through any molecule along the CD200R signaling cascade that results in modulation of Ras/Raf/MAPK, including Dok1, Dok2, SHIP, and GAP, CD200R-mediated medical conditions include (but are not limited to) inflammatory conditions, such as autoimmune diseases, multiple sclerosis and arthritis, allergy, asthma, transplant rejection, prevention of miscarriage, peripheral pain associated with inflammation, graft-host rejection, and cancer.

As used herein the phrase “effective amount” of a composition of the present invention is an amount that will ameliorate one or more of the parameters that characterize CD200R-mediated medical conditions.

As used herein an “agonist of CD200R signaling” is a substance that enhances CD200R signaling and can include, but is not limited to, soluble CD200 ligand, a small molecule, agonistic antibody, and the like. Such agonists should bind to CD200R signaling molecules, in particular, Dok proteins.

As used herein an “antagonist of CD200R signaling” is a substance that inhibits CD200R signaling and can include, but is not limited to, a small molecule, antagonistic antibody, and the like. Such antagonists should bind to CD200R signaling molecules, in particular, Dok proteins.

The present invention may be better understood by reference to the following non-limiting examples, which are provided as exemplary of the invention. These examples are presented in order to more fully illustrate the preferred embodiments of the invention and they should in no way be construed as limiting the scope of the invention.

### EXAMPLES

Materials and General Methods

Some of the standard methods are described or referenced, e.g., in Maniatis, et al. (1982) Molecular Cloning, A Laboratory Manual, Cold Spring Harbor Laboratory, Cold Spring Harbor Press; Sambrook, et al. (1989) Molecular Cloning: A Laboratory Manual (2d ed.), vols. 1-3, CSH Press, NY; Ausubel, et al., Biology, Greene Publishing Associates, Brooklyn, N.Y.; or Ausubel, et al. (1987 and Supplements) Current Protocols in Molecular Biology, Greene and Wiley, New York; Innis, et al (eds.) (1990) PCR Protocols: A Guide to Methods and Applications, Academic Press, N.Y. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification” in Methods in Enzymology vol. 182, and other volumes in this series; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See, e.g., Hochuli (1989) Chemische Industrie 12:69-70; Hochuli (1990) “Purification of Recombinant Proteins with Metal Chelate Absorbent” in Setlow (ed.) Genetic Engineering, Principle and Methods 12:87-98, Plenum Press, N.Y.; and Crowe, et al. (1992) OIAexpress: The High Level Expression & Protein Purification System QIAGEN, Inc., Chatsworth, Calif.

Cell culture techniques are described in Doyle, et al. (eds.) (1994) Cell and Tissue Culture Laboratory Procedures, John Wiley and Sons, NY.

In Vitro Binding Studies of PTB Domain Proteins and CD200R

To determine which PTB domain proteins might bind to CD200R, an in vitro assay was executed utilizing biotinylated peptides corresponding to the three tyrosine residues of CD200R: Y286, Y289, and Y297 which were phosphorylated in all possible combinations. These peptide constructs are listed in Table 1 with the symbol * indicating phosphorylated residues.

TABLE 1
Biotinylated peptides corresponding to portion of CD200R's
cytoplasmic tail.

| Peptide No. | Peptide |
|---|---|
| 1 | Biotin----Y--Y----NPLY---- |
| 2 | Biotin----Y*--Y----NPLY---- |
| 3 | Biotin----Y--Y*----NPLY---- |
| 4 | Biotin----Y--Y----NPLY*---- |
| 5 | Biotin----Y*--Y*----NPLY---- |
| 6 | Biotin----Y*--Y----NPLY*---- |
| 7 | Biotin----Y--Y*----NPLY*---- |
| 8 | Biotin----Y*--Y*----NPLY*---- |

In brief, cells expressing CD200R were stimulated with cellular-Ig (control) or CD200-Ig (an agonist CD200 antibody) and then lysed. Cell lysates were incubated with biotinylated peptides and avidin-agarose beads. After washing, the protein complexes were subjected to SDS-PAGE and Western blot analysis. This assay demonstrated that among the PTB domain proteins assayed, Dok1, Dok2, and Shc bind to phosporyated peptides. In particular, Dok1, Dok2, and Shc bind to peptide Nos. 4 and 6. Dok 2 and Shc also bind to peptide No. 7. Co-immunoprecipitation experiments further confirmed that Dok1 and Dok2 bind to CD200R.

In Vitro Cross-Linking Studies of Dok

Briefly, mouse bone marrow derived mast cells (e.g., cell line WTMC, DT733) were employed to assess the CD200R signaling pathway. Co-crosslinked Fc-gamma-RIIB with Fc-epsilon-RI stimulated Dok1 tyrosine phosphorylation and subsequent association with SHIP and RasGAP. Overexpression of Dok1 in mast cells line RBL-2H3 inhbited Fc-epsilon-RI-mediated Ras/Rafl/Erk signaling and the de novo synthesis of TNF-alpha.

Recombinant Protein, Cytokines and Antibodies

CD200-mIg fusion protein and control mIg were generated as described in Cherwinski et al. (2003) J. Immunol 171:3034-46. Recombinant mouse stem cell factor was purchased from PeproTech (Rocky Hill, N.J.). Rabbit polyclonal anti-Shc, Dok2, SHIP antibody and anti-phosphotyrosine mAb(4G10) were obtained from Upstate Biotechnology, Inc. (Lake Placid, N.Y.). Rabbit polyclonal anti-Dok1 antibodies were purchased from Santa Cruz Biotechnology, Inc. (Santa Cruz, Calif.). Anti-dual phosphorylated MAPK antibodies (ERK, JNK and p38 MAPK), control anti-MAPK, anti-Myc antibody and MEK inhibitor U0126 were obtained from Cell Signaling (Beverly, Mass.). Monoclonal anti-Ras, Dok1, Dok2, RasGAP, PE-labeled anti-Kit antibody and the isotype-matched control antibody were purchased from Pharmingen.

Anti-CD200R antibody (DX109) and anti-CD200RLa antibody (DX89) were made by immunizing six week old female Lewis rats six times with 20 μg each of CD200R-Ig and CD200La-Ig fusion protein, respectively, consisting of the extracellular domain of either murine CD200R or CD200RLa fused to the F_{c} region of human IgG₁ (Hock et al., 2000 Science 290:1768-1771) in Freund's adjuvant intraperitoneally over four months and one intravenous injection 3 days prior to the fusion. Splenocytes were fused with SP2/0 myeloma cells to produce hybridomas using standard techniques. Hybridoma supernatants were screened for their ability to bind to BaF3 cells expressing a cell surface myc tagged murine CD200R or CD200RLa by flow cytometry as a primary screen, and secondly by staining the murine mast cell line WTMC using a goat anti-rat PE conjugated secondary antibody (Caltag, Burlingame, Calif.). The isotype of DX109 and DX89 were determined to be IgG₁ and IgM, respectively, using a rat monoclonal isotyping kit (Zymed, So. San Francisco, Calif.).

Cell Culture, Transduction and Surface Staining

Mouse bone marrow-derived mast cells (WTMCs) were generated from bone marrow of 2-3 week old C57BL 6 mice as previously described in Cherwinski et al. supra. In brief bone marrow cells were cultured in DMEM (BioWittaker, Walkersville, Md.) supplemented with 10% Fetal calf serum (FCS, HyClone, Logan, Utah), 1.0 mM sodium pyruvate, 0.1 mM non-essential amino acids, 0.3 mg/ml L-glutamine, 20 mM Hepes, 50 μM 2-mercaptoethanol, 50 ng/ml recombinant stem cell factor (PeproTech, Rocky Hill, N.J.) and recombinant murine IL-3 (provided by K. Moore, DNAX). After one week the non-adherent cells were removed and plated in fresh media. After two weeks the cells were cultured with RPMI (Bio Whittaker) containing the same supplements as above with the addition of 5 ng/ml recombinant maurine IL-4 (provided by S. Menon, DNAX) but without stem cell factor. After 30 days from the start of culture, cells were >95% positive for CD117 and F_{c}εR.

Mast cells overexpressing CD200R and CD200RLa (DT733) were generated by retroviral transduction of WTMCs. A cDNA containing the CD8 leader segment followed by the Flag epitope (DYKDDDDK) and joined to the extracellular, transmembrane and cytoplasmic domains of mouse CD200RLa was subcloned into the pMXneo retroviral vector (Onihsi, M., et al. (1996) Exp. Hematol. 24:324). Plasmid DNA was transfected into Pheonix ecotropic retrovirus packaging cells (a gift from G. Nolan, Stanford University) using Lipofectamine (Gibco-BRL). Two days later, WTMCs were infected by co-culture with the transfected packaging cell line. After 30 hours, the non-adherent WTMCs were removed and put into fresh media and after 72 hours were switched to selection media containing 1 mg/ml G418 (Roche Molecular Biochemicals, Indianapolis, Ind.). Cells were sorted for CD200RLa expression using the anti-Flag antibody M2 (SIGMA). A cDNA containing the CD8-leader segment followed by the c-myc epitope tag (EQKLISEEDL) and joined to the extracellular, transmembrane, and cytoplasmic regions of mouse CD200R (Wright, G. J., et al. (2000) Immunity 13(2): 233-42) was subcloned into the retroviral vector pMXneo. The resultant construct was then introduced into the mast cell transfectant expressing the Flag-tagged CD200RLa by retroviral infection as described above. Cells were sorted for cell surface CD200R expression using the anti-myc antibody 9E10. Cells ≧95% positive and having similar levels of expression for both the Flag tag and the c-myc tag were used for the biochemical analyses and degranulation assays.

To measure surface expression of CD200R and c-Kit in mast cells, normal growing WTMC and DT733 cells were washed once with PBS and stained with either FITC-conjugated anti-CD200R (DX109, rat IgG1) or PE-conjugated anti-Kit antibody. After incubation at 4° C. for 20 min, cells were washed twice in PBS with 0.5% BSA and analyzed on a FACScan flow cytometer (Becton Dicknson, Mountain View, Calif.).

Cell Stimulation, Degranulation Assay and Cytokine ELISA

Mast cell degranulation was determined using a hexosaminidase release assay as previously described. The degranulation was triggered by DX89 antibody which binds to activating receptor CD200RLa. TNT-α and IL-13 were measured by ELISA kits (R&D Systems, Minneapolis, Minn.), according to the manufacturer's instruction.

Immunoprecipitation and Immunoblotting

Mast cells (1-2×10⁷ cells/ml) were stimulated at 37° C. with control Ig or CD200-Ig (3 ug/ml) for various time as described in figure legend. Cells were then rinsed once with ice-cold PBS containing 1 mM Na₃VO₄ and lysed in lysis buffer (50 mM Tris-HCl, pH 8.0, 150 mM NaCl, 1% Nonidet P-40, 10% glycerol, 5 mM EGTA, 50 nmM NaF, 1 mM Na₃VO₄, plus protease inhibitor cocktails) for 20 min on ice. Lysates were clarified at 14,000 rpm for 10 min. The protein concentration of the supernatant was determined by Bio-Rad protein assay kit (Bio-Rad, Hercules, Calif.). Equal amounts of protein were analyzed by Nu-PAGE (Invitrogen, Calsbard, Calif.) and Western blotting. For Western blotting, primary antibodies were detected with horseradish peroxidase-conjugated secondary antibodies and chemoilluminescence (Pierce, Arlington, Ill.). For immunoprecipitations, antibodies were incubated with 0.5-1 mg of cell lysate for 2 hr at 4° C. The immune complexes were recovered by incubation with protein A-agarose beads or protein G Plus-agarose beads (Santa Cruz Biotech, Santa Cruz, Calif.) for 1 h at 4° C. After washing three times in lysis buffer and once in PBS containing 1 mM Na₃VO₄, the immune complexes were dissociated in SDS sample buffer. The samples were analyzed by Nu-PAGE and Western blotting as described above.

Ras Activation Assay

Ras activation was measured using Ras activation assay kit (Upstate Biotech, Lake Placid, N.Y.) according to the manufacturer's instruction. Briefly, following stimulation cells were lysed. CTP-bound Ras was precipitated by GST fusion protein (containing the Ras binding domain of Raf-1 bound to glutathione-agarose). GTP-bound Ras was detected by Western blot.

Screening Systems and Methods

The present invention allows for the discovery of selective agonists and antagonists of the CD200R signaling that may be useful in the treatment and management CD200R-mediated medical conditions. Thus, agents may be screened for agonist or antagonist activity of CD200R signaling. Essentially, these systems provide methods for bringing together a CD200R, an agent to be tested for the presence of a agonist or antagonist activity. As CD200R inhibits Ras/Raf/MAPK activation, in the case of an agent to be tested for CD200R agonist activity, the cell must first be activated (e.g., by IgE).

Pharmaceutical Compositions

The CD200R agonists and antagonists of this invention can be used therapeutically to enhance or inhibit the activity of CD200R, respectively, to treat any CD200R-mediated medical condition. The dosage regimen involved in a therapeutic application will be determined by the attending physician, considering various factors which may modify the action of the therapeutic substance, e.g., the condition, body weight, sex and diet of the patient, time of administration and other clinical factors.

Typical protocols for the therapeutic administration of such substances are well known in the art. Administration of the pharmaceutical compositions of the present invention is typically by parenteral, intraperitoneal, intravenous, subcutaneous, intramuscular injection, infusion or any other acceptable systemic method. Often, treatment dosages are titrated upward from a low level to optimize safety and efficacy.

Dosages will be adjusted to account for the smaller molecular sizes and possibly decreased half-lives (clearance times) following administration. It will be appreciated by those skilled in the art, however, that the CD200R agonists and antagonists of the present invention encompass antibodies or binding fragments thereof, small organic molecules, as well as ligand analogs, which can be identified using the methods of the present invention.

Although the compositions of this invention could be administered in simple solution, they are more typically used in combination with other materials such as carriers, preferably pharmaceutical carriers. Useful pharmaceutical carriers can be any compatible, non-toxic substances suitable for delivering the compositions of the present invention to a patient. Sterile water, alcohol, fats, waxes, and inert solids may be included in a carrier. Pharmaceutically acceptable adjuvants (buffering agents, dispersing agents) may also be incorporated into the pharmaceutical composition. Generally, compositions useful for parenteral administration of such drugs are well known; e.g., Remington's Pharmaceutical Science, 17th Ed. (Mack Publishing Company, Easton, Pa., 1990). Alternatively, compositions of the present invention may be introduced into a patient's body by unplantable drug delivery systems [Urquhart et al., Ann. Rev. Pharmacol. Toxicol. 24:199 (1984)].

Pharmaceutically acceptable carriers are determined in part by the particular composition being administered, as well as by the particular method used to administer the composition. Accordingly, there are a wide variety of pharmaceutical composition formulations suitable for the present invention.

Formulations suitable for parenteral administration, for example, by intravenous, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Therapeutic formulations may be administered in many conventional dosage formulations. Formulations typically comprise at least one active ingredient, together with one or more pharmaceutically acceptable carriers. Formulations may include those suitable for oral, rectal, nasal or parenteral (including subcutaneous, intramuscular, intravenous and intradermal) administration.

The formulations may conveniently be presented in unit dosage form and may be prepared by any method well known in the art of pharmacy. See, e.g., Gilman et al. (eds.) (1990), The Pharmacological Bases of Therapeutics, 8th Ed., Pergamon Press; and Remington 's Pharmaceutical Sciences, supra, Easton, Pa.; Avis et al. (eds.) (1993) Pharmaceutical Dosage Forms: Parenteral Medications Dekker, N.Y.; and Lieberman et al. (eds) (1990) Pharmaceutical Dosage Forms: Tablets Dekker, N.Y.; and Lieberman et al. (eds.) 1990), Pharmaceutical Dosage Forms: Disperse Systems Dekker, N.Y.

Many modifications and variations of this invention can be made without departing from its spirit and scope, as will be apparent to those skilled in the art. The specific embodiments described herein are offered by way of example only, and the present invention is to be limited only by the terms of the appended claims, together with the full scope of equivalents to which such claims are entitled. Numerous references are cited in the specification, the disclosures of which are incorporated by reference in their entireties.

What is claimed is:

1. A method for modulating CD200R signaling in a mammal, comprising administering to the mammal an effective amount of an agent that is effective to modulate CD200R signaling in the mammal.

2. The method of claim 1 wherein CD200R signaling is measured by assaying TNT-alpha production.

3. The method of claim 1 wherein CD200R signaling is measured by assaying mast cell degranulation in a sample taken from the mammal.

4. The method of claim 1 wherein CD200R signaling is inhibited by a Dok protein antagonist.

5. The method of claim 1 wherein CD200R signaling is enhanced by a Dok protein agonist.

6. The method of claim 1 wherein the agent is administered at a frequency and for a duration sufficient to inhibit cytokine production in the mammal at a level lesser than the cytokine production measured prior to the step of administering the agent.

7. The method of claim 1 wherein the agent is administered at a frequency and for a duration sufficient to enhance cytokine production in the mammal at a level greater than the cytokine production measured prior to the step of administering the agent.

8. A method for treating CD200-mediated medical conditions in a mammal, comprising administering to the mammal an effective amount of an agonist of CD200R signaling.

9. The method of claim 3, wherein the agonist is a Dok protein agonist.

10. A method for treating CD200-mediated medical conditions in a mammal, comprising administering to the mammal an effective amount of an antagonist of CD200R signaling.

11. The method of claim 10, wherein the antagonist is a Dok protein antagonist.

12. A method of modulating physiology of a cell by contacting the cell with an agonist or antagonist of CD200R signaling.

13. The method of claim 12, wherein the agonist is a Dok protein agonist.

14. The method of claim 12, wherein the antagonist is a Dok protein antagonist.
